(19) United States
(12) Patent Application Publication
(10) Pub. No.: US 2018/0353549 A1
(43) Pub. Date: Dec. 13, 2018

(54) COMPOSITIONS AND METHOD FOR TREATING AND PREVENTING BLEEDING AND LUNG INJURIES AND DISEASES

(71) Applicant: Vet-Stem, Inc., Poway, CA (US)

(72) Inventor: Robert Harman, Ramona, CA (US)

(21) Appl. No.: 15/781,084

(22) PCT Filed: Dec. 5, 2016
(86) PCT No.: PCT/US16/64985

§ 371 Date: Jun. 1, 2018

Related U.S. Application Data
(60) Provisional application No. 62/263,501, filed on Dec. 4, 2015.

(51) Int. Cl. A61K 35/28; A61P 7/04; A61P 11/00; A61K 45/06

(52) U.S. Cl. CPC A61K 35/28; A61P 7/04; A61K 9/0019; A61K 45/06; A61P 11/00

(57) ABSTRACT

The present invention provides methods for treating and preventing bleeding and lung injuries, such as exercise-induced pulmonary hemorrhage, using compositions comprising stem cells and/or stem cell derived factors.

### CROSS REFERENCE TO RELATED APPLICATIONS

This application claims priority to U.S. Provisional Application No. 62/263,501, filed on Dec. 4, 2015, which is incorporated by reference herein in its entirety.

### BACKGROUND

### Field

The present invention is directed to methods for treating and preventing injuries and diseases, such as bleeding injuries or lung injuries, e.g., exercise-induced pulmonary hemorrhage, comprising providing to a subject in need thereof an effective amount of stem cells or stem cell derived factors.

### Description of the Related Art

A variety of bleeding or lung injuries are associated with damage to blood vessels or inflammation. For example, exercise induced pulmonary hemorrhage (EIPH), also known as “bleeding” or a “bleeding attack”, refers to the presence of blood in the airways of the lung in association with exercise. EIPH is common in horses undertaking intense exercise, and it has also been reported in human athletes, racing camels and racing greyhounds. Horses suffering from EIPH may also be referred to as “bleeders” or as having “broken a blood vessel”. EIPH is properly diagnosed by an endoscopic examination of the airways performed following exercise. However, horses may also show bleeding at the nostrils after exercise, which is known as epistaxis. The lung can also be injured and bleeding occur by trauma as during severe exercise or accidental trauma.

Furosemide (Lasix) has been used extensively to minimise EIPH, but it is believed to be ineffective in 50% of cases. In addition, there are undesirable side-effects associated with chronic use of Lasix, which include hypokalemia and hypomagnesemia. The use of Lasix in competing horses is prohibited in some countries and it is regarded as a banned substance by the International Olympic Committee and many jurisdictions are in discussions about banning Lasix.

Thermal injury and smoke inhalation, e.g., resulting from exposure to fire, can cause local and diffuse lesions, e.g., in the throat and lungs of exposed animals. Massive tissue edema may occur, which can be a challenge to manage as well as creating organ dysfunction at distant sites. Further complications of severely affected patients are varied and include life-threatening sepsis.

Thus, there is clearly a need in the art for more effective and safer treatments for EIPH and other diseases and injuries, including bleeding disorders and diseases and injuries to the lung.

### BRIEF SUMMARY

The present invention provides methods, compositions and kits for treating and preventing disease and injury in a mammal.

In one embodiment, the present invention provides a method for treating or preventing an injury or disease associated with damage to blood vessels, inflammation, and/or fibrosis in a mammal in need thereof, comprising providing to the mammal an effective amount of a pharmaceutical composition comprising one or more stem cells, or one or more stem cell derived factors. In certain embodiments, the mammal is a horse, human, camel or dog. In particular embodiments, the stem cells were obtained from the mammal or from a donor animal.

In particular embodiments, the pharmaceutical composition comprises a stromal vascular fraction comprising stem cells, isolated stem cells, or stem cell derived factors. In particular embodiments, the stem cells are derived from adipose tissue.

In particular embodiments, the pharmaceutical composition is administered intravenously, orally, nasally, or by inhalation. In particular embodiments, the pharmaceutical composition further comprises one or more additional active agent for the treatment of the lung injury. In some embodiments, the one or more additional active agent comprises a non-steroidal anti-inflammatory agent or a steroid.

In various embodiments, the injury or disease treated or prevented is an injury or disease of a lung and/or major airways. In certain embodiments, the lung injury is exercise induced pulmonary hemorrhage (EIPH), chronic obstructive pulmonary disorder (COPD), lung fibrosis, smoke inhalation, other toxic inhalation, or pneumonia infection.

### DETAILED DESCRIPTION

In the following description, certain specific details are set forth in order to provide a thorough understanding of various embodiments of the invention. However, one skilled in the art will understand that the invention may be practiced without these details.

### Definitions

Unless defined otherwise, all technical and scientific terms used herein have the same meaning as commonly understood by those of ordinary skill in the art to which the invention belongs. For the purposes of the present invention, the following terms are defined below.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

A “decreased” or “reduced” or “lesser” amount is typically a “statistically significant” amount, and may include a decrease that is about 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times lower (e.g., 100, 500, 1000 times) an amount or level described herein. In particular embodiments, it indicates a decrease of at least 10%, at least 20%, at least 30%, at least 40%, at least 50%, at least 60%, at least 70%, at least 80%, or at least 90% (including all integers and decimal points in between, e.g., 15%, 26%, etc.) as compared to the reference amount.

Reference throughout this specification to “an embodiment” or “one embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times greater (e.g., 100, 500, 1000 times) (including all integers and decimal points in between and above 1, e.g., 2.1, 2.2, 2.3, 2.4, etc.) an amount or level described herein. In particular embodiments, it indicates an increase of at least 10%, at least 20%, at least 30%, at least 40%, at least 50%, at least 60%, at least 70%, at least 80%, at least 90%, at least 100%, at least 150%, at least 200%, at least 500%, or at least 1000% (including all integers and decimal points in between, e.g., 15%, 26%, etc.) as compared to the reference amount.

By “isolated” is meant material that is substantially or essentially free from components that normally accompany it in its native state. For example, an “isolated polynucleotide,” as used herein, includes a polynucleotide that has been purified from the sequences that flank it in its naturally-occurring state, e.g., a DNA fragment which has been removed from the sequences that are normally adjacent to the fragment. Alternatively, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances.

By “obtained from” is meant that a sample such as, for example, a biological sample or tumor sample, is isolated from, or derived from, a particular source, such as a desired organism (e.g., subject) or a specific tissue within a desired organism. For example, a biological sample may be obtained from a subject. “Derived from” or “obtained from” can also refer to the source of a biological sample or tumor tissue sample.

A “subject,” as used herein, includes any animal that exhibits a symptom, or is at risk for exhibiting a symptom, which can be treated according to the invention. Suitable subjects (patients) include mammals (such as humans and non-human primates), laboratory animals (such as mouse, rat, rabbit, or guinea pig), farm animals, sport or racing animals (such as race horses or camels), and domestic animals or pets (such as a cat or dog).

“Treatment” or “treating,” as used herein, includes any desirable effect on the symptoms or pathology of a disease or condition, e.g., EIPH, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Illustrative markers of clinical improvement will be apparent to persons skilled in the art.

“Prevention” or “preventing,” as used herein, includes delaying or inhibiting the onset or progression of symptoms or pathology of a disease or condition, e.g., EIPH, and may include even minimal changes or improvements in one or more measurable markers of the disease or condition being treated. “Prevent” or “preventing” does not necessarily indicate complete prevention of the onset or progression of the disease or condition, or associated symptoms thereof.

The term “stem cell”, as used herein, may comprise hematopoietic or non-hematopoietic cells which exist in almost all tissues and have the capacity of self-renewal and the potential to differentiate into multiple cell types.

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (3rd Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5th Ed. Hoboken N.J., John Wiley & Sons; B. Perbal, A Practical Guide to Molecular Cloning (3rd Edition 2010); Farrell, R., RNA Methodologies: A Laboratory Guide for Isolation and Characterization (3rd Edition 2005), Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-3, 4-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, (2002, Cold Spring Harbor Laboratory, ISBN 0-87969-630-3).

### DETAILED DESCRIPTION

The present invention includes methods of treating or preventing injuries and disease using stem cells, e.g., preparations of stromal vascular fraction from adipose tissue, and/or stem cell derived factors. In particular embodiments, the methods are used to treat injuries and disease associated with damage to blood vessels, inflammation, and/or fibrosis. In certain embodiments, both stem cells and stem cell derived factors are provided to the subject. In particular embodiments, the treatment stabilizes blood vessel integrity and/or treats or prevents inflammation and/or fibrosis. In particular embodiments, the injury or disease is associated with bleeding.

In particular embodiments, the injury or disease is an injury or disease of the lung or affecting the lung. Examples of lung injuries and diseases that may be treated according to methods of the present invention include, but are not limited to: exercise induced pulmonary hemorrhage (EIPH), chronic obstructive pulmonary disorder (COPD), lung fibrosis, smoke inhalations, other toxic inhalations, pneumonia infection (bacterial or viral), toxins, mold, other bacterial infection, other viral infection, and traumatic injury. Other injuries or diseases that may be treated include, e.g., nasopharyngeal cicatrix. In particular embodiments, methods of the present invention comprise providing a composition comprising stem cells to a subject being treated intravenously or systemically.

Exercise-induced pulmonary hemorrhage (EIPH) occurs in the majority of Thoroughbred and Standardbred racehorses, as well as other breeds that are required to perform very strenuous exercise in their athletic disciplines (i.e., barrel racing Quarter Horses). EIPH has also been reported in human athletes, racing camels and racing greyhounds. EIPH is bleeding that occurs from the lungs of horses during exercise, and has been shown to decrease athletic performance, and to gradually worsen over time with ongoing athletic performance at the same intensity level. A variety of different causes of EIPH have been proposed. These include high pulmonary vascular pressure, upper airway obstruction, mechanical trauma, lower airway obstruction, inflammation, abnormalities of blood coagulation, inhomogeneity of ventilation and locomotory trauma.

During the past four decades, furosemide has been used prophylactically before racing, to try to prevent EIPH. Studies have shown that furosemide temporarily decreases pulmonary vascular pressures during strenuous exercise, which may decrease the likelihood of capillary stress failure, and therefore reduce the likelihood of hemorrhage in the lungs.

It is known that EIPH causes ongoing structural damage to the lungs. Studies have shown fibrosis and vascular remodeling of the caudodorsal lung fields, as well as venous remodeling and occasional bronchiolar damage in horses with EIPH. This, in turn, causes pulmonary hypertension that can lead to capillary stress failure and hemorrhage into the lung from the damaged pulmonary capillaries. Lower airway inflammation may also play a secondary role in the development of EIPH. To this date there is no known “treatment” for EIPH, and current prophylactic therapies cannot stop the progression of the condition. The present invention provided compositions and methods for treating EIPH.

The present invention is based, in part, on the observation that treating animals having EIPH or smoke inhalation injury with adipose-derived stem cells (ASCs) reduced the disease or injury, or associated symptoms. As described in the accompanying Example, six horses with repeated epistaxis and endoscopic confirmation of lung bleeding, while racing on prophylactic medications, were treated with ASCs. Prior to treatment with ASCs, all six of the horses had had obvious epistaxis after racing and veterinary confirmation of lung bleeding by endoscope, despite having received pre-race prophylactic medications (5/6 horses). All six horses were barrel racing quarter horses. Following treatment with ASCs, none of the six horses had epistaxis post-race. Three of the six horses (50%) were confirmed Grade 0 EIPH (no bleeding) by post-race TBS examination. Two horses (33%) were confirmed Grade 1 EIPH, and one horse (17%) was confirmed Grade 2 EIPH on post-race TBS examinations This data demonstrates that stem cells may be used to treat EIPH.

In particular embodiments, methods of the present invention may be used to treat animals diagnosed with or suspected of having EIPH. Such animals include, but are not limited to, mammals, such as humans, horses, dogs and camels. In particular embodiments, the animal is a racing animal or an animal that undergoes strenuous activity on a regular basis.

EIPH may be diagnosed in an animal through a variety of methods, including but not limited to: visual assessment, e.g., blood in the nostril; endoscopy, i.e., endoscopic examination of the trachea and large airways following exercise (e.g., around 30-60 minutes after exercise); bronchoalveolar lavage (BAL), e.g., to determine if blood is present in smaller airways of the lung; cytopathology, e.g., to determine the presence of high levels of red blood cells and/or hemosiderophages; radiography of the chest; and pulmonary scintigraphy, e.g., to detect alteration in the perfusion and/or ventilation of the dorso-caudal lung. For endoscopy, the amount of blood visible in the trachea at the time of examination is most commonly graded on a 0 (no blood) to 4 (airways awash with blood) scale.

In particular embodiments, methods of the present invention may be used to treat animals suffering from smoke inhalation or a related injury, such as inflammation, burning, or scarring of the respiratory tract or lungs.

### Methods of Treatment

In particular embodiments, the present invention includes methods of treating or preventing a disease or injury, e.g., EIPH, in a subject in need thereof, comprising providing to the subject an effective amount of a pharmaceutical composition comprising stem cells. In particular embodiments, the present invention includes methods of treating or preventing a disease or injury, e.g., EIPH, in a subject in need thereof, comprising providing to the subject an effective amount of a pharmaceutical composition comprising stem cell derived factors. In particular embodiments, the present invention includes methods of treating or preventing a disease or injury, e.g., EIPH, in a subject in need thereof, comprising providing to the subject an effective amount of a pharmaceutical composition comprising stem cells and a pharmaceutical composition comprising stem cell derived factors. In certain embodiments, the stem cells and the stem cell derived factors are present in the same pharmaceutical composition.

Stem cells, including SVF, stem cell derived factors, and related compositions may be provided to a subject by a variety of different means. In certain embodiments, they are provided locally, e.g., to a site of actual or potential injury. In one embodiment, they are provided using a syringe to inject the compositions at a site of possible or actual injury or disease. In other embodiments, they are provided systemically. In one embodiment, they are administered to the bloodstream intravenously or intra-arterially. Accordingly, the invention includes providing a cell population or composition of the invention via any known and available method or route, including but not limited to oral, parenteral, intravenous, intra-arterial, intranasal, inhalation, and intramuscular administration.

The present invention includes autologous, allogeneic, syngeneic, and xenogeneic treatments, so the stem cells or stem cell factors provided to the subject may comprise stem cells or stem cell factors obtained from the subject or a donor (or cells derived from the subject or a donor). In particular embodiments, the stem cells were obtained from the same species of animal as the subject. In certain embodiments, the stem cells or stem cell factors were obtained from a different species of animal as the subject.

“Stem cell derived factors” are compounds, e.g., polypeptides, secreted by stem cells, and include, but are not limited to, cytokines, growth factors, and chemokines. Illustrative stem cell derived growth factors include, but are not limited to, hepatocyte growth factor (HGF), basic fibroblast growth factor (bFGF), granulocyte-colony stimulating factor (G-CSF), granulocyte macrophage-colony stimulating factor (GM-CSF), interleukin-7 (IL-7), platelet derived growth factor BB (PDGF-bb) and vascular endothelial growth factor (VEGF). Examples of stem cell derived anti-inflammatory cytokines include, but are not limited to, IL-1Ra, IL-2, IL-4, IL-6, IL-10 and IL-13. Examples of stem cell derived chemokines include, but are not limited to, Eotaxin, MIP-1α, MIP-1β and RANTES.

In certain embodiments, each of the one or more stem cell derived factors is selected from the group consisting of: adiponectin (Acrp30), Agouti-related peptide (AgRP), angiopoietin-2, basic fibroblast growth factor (bFGF), BTC, epidermal growth factor receptor (EGF-R), FAS, fibroblast growth factor (FGF)-4, FGF-9, granulocyte colony stimulating factor (G-CSF), glucocorticoid-induced tumor necrosis factor receptor (GITR), GITR-ligand, chemokine C—X—C motif ligand (GRO), hepatocyte growth factor (HGF), intercellular adhesion molecule (ICAM)-3, insulin-like growth factor (IGF)-1SR, IGF-binding protein (IGFBP)-3, IGFBP-6, interleukin-2 receptor alpha (IL-2Rα), interleukin-6 receptor (IL-6R), interleukin (IL)-8, IL-11, IL-12p40, IL-17, lymphotaktin, membrane cofactor protein (MCP)-1, macrophage migration inhibitory factor (MIF), macrophage inflammatory protein (MIP)-1.alpha., MIP-1β, MIP-3β, macrophase stimulating protein (MSP) α, neurotrophin (NT)-4, oncostatin M, osteoprotegerin, phosphatidylinositol-glycan biosynthesis class F (PIGF), sgp130, soluble tumor necrosis factor receptor type 2 (sTNF RH), tissue inhibitor of metalloproteinase (TIMP)-1, TIMP-2, TNF-related apoptosis-inducing ligand (TRAIL) receptor 3 (R3), TRAIL R4, urokinase receptor (uPAR), vascular endothelial growth factor (VEGF), and VEGF-D.

Stem cell derived factors include isolated purified stem cell factors, and also include one or more stem cell factors present in conditioned media from cultured stem cells, or a pharmaceutical composition comprising one or more stem cell derived factors. Stem cell factors may also be produced recombinantly. A variety of purified or recombinantly produced stem cell factors are commercially available. Accordingly, methods of the present invention include providing to a subject one or more purified stem cell factors or recombinantly produce stem cell factors, conditioned media from cultured stem cells, stem cell factors isolated from conditioned media from cultured stem cells, and pharmaceutical compositions comprising any of these.

In one specific embodiment, a method of treatment comprises providing to a mammal, e.g., a horse, diagnosed with EIPH, an effective amount of a pharmaceutical composition comprising stem cells obtained from the same animal. In particular embodiments, the mammal is a Quarter Horse. In particular embodiments, the pharmaceutical composition is provided to the mammal intravenously. In certain embodiments, the pharmaceutical composition comprises stromal vascular fraction cells, which include stem cells. In particular embodiments, the horse is provided with between 500,000 to 50,000,000 stromal vascular fraction cells. In certain embodiments, the animal is provided with between 1,000,000 and 20,000,000 stromal vascular fraction cells.

The development of suitable dosing and treatment regimens for using the cell populations, stem cell factors, and compositions described herein in a variety of treatment regimens, including e.g., oral, parenteral, intravenous, intranasal, inhalation, and intramuscular administration and the appropriate formulation, will again be driven in large part by the type of animal being treated and the route of administration. The determination of suitable dosages and treatment regimens may be readily accomplished based upon information provided herein and generally known in the art. In certain embodiments, an animal, e.g., horse, is administered about 1 million to about 100 million, 1 million to about 50 million cells, e.g., in SVF or cultured cells. In particular embodiments, a animal, e.g., horse, is administered about 1 million to about 20 million cells, about 1 million to about 15 million cells, about 2 million to about 15 million cells, about 4 million to about 15 million cells, less than 50 million cells, less than 25 million cells, less than 20 million cells, less than 15 million cells, less than 10 million cells, less than 5 million cells, less than 2 million cells, or less than 1 million cells per treatment. In certain embodiments, an animal is administered SVF comprising about 1-20 million or about 2.8-16.5 million nucleated cells, and in certain embodiments, an animal is administered cultured ASCs comprising about 10-50 million or about 15.4-30.8 million stem cells.

Treatment may comprise a single treatment or multiple treatments. In certain embodiments, an animal is provided with two or more treatments. In certain embodiments, an animal is provided with one or two treatments, wherein each treatment comprises administering to the animal about 1 million to about 20 million cells, about 1 million to about 15 million cells, about 2 million to about 15 million cells, about 4 million to about 15 million cells, less than 50 million cells, less than 25 million cells, less than 20 million cells, less than 15 million cells, less than 10 million cells, less than 5 million cells, less than 2 million cells, or less than 1 million cells per treatment. In particular embodiments, the treatments are spaced apart by at least or about one day, two days, one week, two weeks, one month, two months, four months, or six months. In particular embodiments, e.g., preventative purposes, it is contemplated that treatment occurs prior to a stress that might potentially cause or exaggerate EIPH, such as, e.g., an animal race (e.g., camel or horse race).

Subjects being treated according to the invention may also be treated with one or more additional therapeutic agents. The one or more additional therapeutic agents may be administered separately, or it may be present in the pharmaceutical composition comprising stem cells or stem cell derived factors. In certain embodiments, the one or more additional agent is selected from: non-steroidal anti-inflammatory drugs (NSAIDs); anti-inflammatories (e.g. corticosteroids), bronchodilators (e.g., ipratropium bromide), anti-hypertensive agents (including nitric oxide donors and phosphodiesterase inhibitors), conjugated estrogens (e.g. Premarin), antifibrinolytics (e.g. aminocaproic acid and tranexamic acid), snake venom, aspirin, vitamin K, bioflavinoids, diuretics (e.g. furosemide, known as Lasix or Salix), concentrated equine serum omega-3 fatty acids.

In particular embodiments, subjects treated according to the present invention show a clinical improvement, e.g., one or more of reduced airway inflammation, e.g., during an episode of EIPH, improved healing of lung tissue, e.g., after an episode of EIPH, or both (as compared to when no treatment is provided), or reduction in airway bleeding. Improved healing can mean taking less time to reach a desired healthy end-state, and/or bringing the lung and/or upper respiratory tract tissue to a healthier final state. In certain embodiments, subjects treated according to the present invention show a reduction or slowing in the progression of a disease, e.g., due to decreased inflammation and/or decreased scarring.

### Compositions Comprising Stem Cells or Stem Cell Derived Factors

In particular embodiments, pharmaceutical compositions comprising stem cells comprise purified stem cells. In other embodiments, they comprise stromal vascular fraction (SVF), which itself includes stem cells. In certain embodiments, they comprise stem cells that have been cultured in vitro, while in other embodiments, the cells have not been cultured. In particular embodiments, the stem cells are used directly after tissue processing or after storage on ice for less than one week or less than 3 days. In other embodiments, they are frozen, e.g., at about −70° C. or about −180° C. and then thawed at some future time before use.

Adipose tissue is a highly vascularized organ containing a dense network of capillary beds surrounding mature adipocytes. Associated with these capillary beds are a number of different cell types. Using collagenase, the connective tissue can be broken down, thereby releasing the cells. Subsequent centrifugation results in floating adipocytes, and pelleted cells, termed the stromal vascular fraction (SVF). The SVF contains high numbers of T regulatory cells and macrophages, as well as endothelial cells and smooth muscle cells. In addition, SVF contains endothelial precursor cells, which may be important in the blood vessel repair. Additionally, the SVF from adipose tissue is a rich source of mesenchymal stem cells (MSCs) containing approximately 500 times more MSCs per gram than bone marrow. MSCs derived from adipose tissue are functionally similar to bone marrow derived MSC (BM-MSC). The relative abundance of MSCs in adipose tissue compared to bone marrow, the relative ease of obtaining large volumes of tissue and the ability to rapidly isolate the SVF, makes adipose tissue an attractive source of MSCs.

Stem cells, including SVF, used according to the present invention may be obtained from any tissue source, including but not limited to adipose tissue, umbilical cord matrix, brain tissue, blood, muscle, bone marrow, tooth tissue and skin. In one embodiment, the tissue is a collagen-based tissue, such as adipose tissue or umbilical cord matrix.

In certain embodiments, a stem cell is of mesodermal origin. Typically, such stem cells retain two or more mesodermal or mesenchymal developmental phenotypes. In particular, such cells have the capacity to develop into mesodermal tissues, such as mature adipose tissue, bone, various tissues of the heart, dermal connective tissue, hemangial tissues, muscle tissues, urogenital tissues, pleural and peritoneal tissues, viscera, mesodermal glandular tissue and stromal tissue. In other embodiment, a stem cell has the capacity to develop into neural ectodermal tissue.

In certain embodiments, a stem cell composition is prepared as described in U.S. Patent Application Publication No. US20070274960.

In particular embodiments, the stem cell composition is a stromal vascular fraction. In particular embodiments, the stem cell composition is prepared as described in Example 1.

In certain embodiments, stromal vascular fraction or stem cells are prepared by processing tissue to release cells from other tissue components.

Tissue may be isolated from a subject or donor by any means available in the art. In certain embodiments, tissue is isolated by lipoaspiration, surgical removal, withdrawal using a needle and syringe, or lipectomy. A variety of additional procedures are described in U.S. Patent Application Publication No. 2003/0161816 A1 and U.S. Pat. Nos. 6,020,196 and 5,744,360. Furthermore, tissue may be isolated from any suitable location on an animal, depending upon the type of tissue being isolated. For example, adipose tissue may be isolated from locations including, but not limited to, the tail head, the omentum or other abdominal location, subcutaneously, the stomach, hips or thighs. As used herein, the tail head region is the general area from the midline lateral and cranial to the insertion of the tail into the body of the animal, extending forward to the area of the loin and the points of the hips. Umbilical cord matrix is typically isolated from the matrix within the umbilical cord, otherwise referred to as Wharton's jelly.

In certain embodiments, a tissue is processed to release cells from other tissue components by any of a variety of different means or combinations thereof. In many embodiments, tissue is physically processed, e.g., by cutting or mincing a tissue sample into smaller pieces. In certain embodiments, tissue is processed by exposure to an enzyme preparation that facilitates the release of cells from other tissue components, while in other embodiments, the processing of tissue does not include exposure to an enzyme that facilitates the release of cells from other tissue components. In one embodiment, the enzyme preparation is a collagenase preparation or comprises collagenase. In related embodiments, the enzyme preparation comprises one or more of trypsin-like, pepsin-like, clostripain, and neutral protease-type enzymes. In some embodiments, it comprises hyaluronidase, or both collagenase and hyaluronidase. Typically, the methods of the invention include processing by one or more of the following procedures: physical cutting, enzymatic treatment, ultrasonic energy treatment, and perfluorocarbon treatment.

In one embodiment, the processing of a tissue comprises physically cutting the tissue into smaller pieces. Cutting may be performed by any means available, including, e.g., the use of scissors, scalpels, razor blades, needles, filters, wires, and other sharp instruments.

In certain embodiments, processing of the tissue includes enzymatic treatment. Typically, enzymatic treatment involves exposing the tissue to one or more enzymes that facilitate the release of cells from other tissue components. Example of such enzymes include matrix metalloproteinases, clostripain, trypsin-like, pepsin-like, neutral protease-type and collagenases. Suitable proteolytic enzymes are described in U.S. Pat. Nos. 5,079,160; 6,589,728; 5,422,261; 5,424,208; and 5,322,790. In one embodiment, a tissue sample is exposed to collagenase at a concentration in a range of 0.01 to 10.0 mg/ml, 0.05 to 10 mg/ml, 0.5 to 2.5 mg/ml, or 0.75 to 2.0 mg/ml, for a time sufficient to release cells from other tissue components. In a related embodiment, the level of collagenase is 0.75 mg/ml (0.075%). The actual usage level may be routinely determined by the skilled artisan for the particular tissue type being digested, and it is further understood that the concentration may vary depending upon the particular source of the enzyme. In particular embodiments, collagenase is used at approximately 0.75 or 0.9 mg/ml (Sigma-Aldrich, Cat. #2674), or 0.75 or 2.0 mg/ml (Serva NB4). Enzymatic treatment may be performed at a variety of different temperatures and time durations, which are understood generally to be inversely correlated to some degree. For example, in one embodiment, collagenase treatment is performed at 37° C. for 2-5 minutes multiple times (with removal of cells after each time period) or as long as 3-4 hours. In one embodiment, the total incubation with enzyme is 20-60 minutes.

In one embodiment, ultrasonic energy is used to process a tissue sample. In a specific embodiment, a transducer is applied to a fluid filled chamber containing the tissue being processed. The energy is applied and dissolution of the tissue occurs. In related embodiment, this procedure is performed separately or in combination with enzymatic treatment. Conditions of the ultrasonic treatment are selected so that adipose tissue is affected without the cells therein being significantly damaged. The use of ultrasonic energy has previously been shown to improve the dissolution of adipose tissue under in vivo procedures relating to lipoaspiration and suitable conditions for in vivo dissolution of adipose tissue have been described in US Patent Application Publication No. 2002/0128592 A1, which conditions may be adapted for the in vitro uses described herein.

In another embodiment, processing of a tissue sample includes treatment with a medically-compatible perfluorocarbon solution. Typically, the adipose tissue is placed into contact with or mixed with the perfluorocarbon solution for sufficient time to generate an emulsion. The perfluorocarbon solution layer is then aspirated, leaving the aqueous layer containing the stem cells. The use of medically-compatible compositions of perfluorocarbons has been reported to aid in the in vivo removal of adipose tissue performed on human subjects (see, e.g., U.S. Pat. No. 6,302,863), and methods and perfluorocarbon solutions described therein may be applied to the in vitro methods of the present invention.

In certain embodiments, released cells are separated from other tissue components after or concurrent with the processing of a tissue sample. As used herein, separation of cells means the release of cells from their normal tissue environment and does not indicate that the cells are purified or isolated from all other tissue components. In certain embodiments, separation of cells comprises separating cells from certain insoluble tissue components, including residual tissue material, such as lipids. Cells may be separated from other tissue components by any means known and available in the art, including, e.g., the use of density gradients, centrifugation, and filtration or combinations thereof. Example of specific methods of purifying cells are known and described in the art, e.g., in U.S. Pat. No. 6,777,231. In certain embodiments, negative separation methods are employed to remove one or more particular types of cells.

In certain embodiments, stem cells, SVF or stem cell derived factors are prepared as described in Blaber et al., Journal of Translational Medicine 2012, 10:172. For each sample, lipoaspirate is enzymatically digested, e.g., with 0.5 mg/mL collagenase (Lomb Scientific, USA) mixed with 0.05 mg/mL of vancomycin (Hospira Australia Pty Ltd, Australia) in a 37° C. water bath for 30 mins with periodic mixing. The digested samples are filtered, e.g., passed through an 800 μm mesh, and centrifuged, e.g., at 1500×g for 5 mins, to obtain the pelleted cells (SVF) and floating adipocytes.

In certain embodiments, the adipocyte and SVF fractions may be washed separately with saline and centrifuged, e.g., at 1500×g for 5 mins. The freshly isolated fractions may be placed into culture to produce conditioned medium comprising stem cell derived factors.

In particular embodiments, to obtain a population of adherent ADSCs, a portion of each SVF pellet obtained may be placed into a T175 cm2 flask containing Standard Media that consisted of Dulbeccos Modified Eagle Medium (DMEM; Invitrogen, USA) supplemented with 10% fetal bovine serum (FBS; Bovogen, Australia) and 1% Penicillin-Streptomycin solution (Invitrogen, USA). Media changes may be performed, e.g., every 3 days. The initial media change results in removal of nonadherent cells. Once the adherent ADS Cs reached about 80% confluency, cells are passaged, e.g., using TrypLE express (Invitrogen, USA).

Cells prepared according to the methods of the invention may be used immediately or stored prior to use. In certain embodiments, cells are isolated from a tissue sample at a geographic location different from the location where the tissue sample was obtained or where the tissue sample is to be provided to a patient. In such circumstances, the purified cells are typically stored prior to shipment to a physician or veterinarian for administration to a patient. The cells may be stored temporarily at approximately 4° C., or the cells may be frozen under liquid nitrogen for long term storage. A variety of methods of freezing cells for long term storage and recovery are known in the art and may be used according to the invention, including freezing cells in a medium comprising fetal bovine serum and dimethylsulfoxide (DMSO).

In certain embodiments, processed cells (or pharmaceutical composition comprising the processed cells, whether previously frozen or not, are placed into a vehicle suitable for administration. For example, processed cells may be placed into a syringe suitable for injection into a subject or via intravenous administration.

In certain embodiments, the processed cells include stem cells, and they can also include other cell types, such as one or more of the following: red blood cells, white blood cells, neutrophils, monocyte/macrophages, fibroblasts, fibroblast-like cells, lymphocytes, and basophils. However, in certain embodiments, the compositions and cell populations do not include lymphocytes (i.e., T or B cells) or have a significantly reduced percentage of lymphocytes as compared to the amount present in peripheral blood. In specific embodiments, the percent of total cells in the purified cell population that are lymphocytes is reduced by at least 50%, 60%, 70%, 80%, 90%, 95%, 99% or 100% as compared to the percent of total cells in the original tissue sample that are lymphocytes. In related embodiments, lymphocytes represent less than 1%, 2%, 5%, 10%, 20%, 30%, 40%, or 50% of the total cells present in the purified cell population. In particular embodiments, the purified cell population does not comprise an appreciable number of lymphocytes. An appreciable number of lymphocytes, as used herein, refers to at least 5% of the cell population being lymphocytes. Since the methods of the invention do not typically include a step of separating stem cells from other processed cells, these additional cells may be present in the originally purified cell population. Alternatively, non-stem cells may be added to the purified cell population at any time prior to administration to a patient.

In further embodiments, the cell populations also include non-cellular tissue components. Such non-cellular components may be soluble factors, or, alternatively, they may be insoluble components, such as lipids, or both. Examples of such non-cellular tissue components include extracellular matrix proteins, proteoglycans, secreted factors, cytokines, growth factors, differentiation-inducing factors, and differentiation-inhibiting factors, or fragments thereof. In one embodiment, the cell populations include collagen, thrombospondin, fibronectin, vitronectin, laminin, or fragments thereof. In a particular embodiment, the cell populations include collagen or fragments thereof. Collagens include, but are not limited to, Type I, Type II, Type III, and Type IV collagen. Again, these additional non-cell components frequently will be present in the originally isolated cell population. However, in certain embodiments, such non-cell components are added to the purified cell population prior to administration to a patient.

In certain embodiments, the processed cells, e.g., SVF, or stem cell derived factors, are present within a pharmaceutical composition adapted for and suitable for delivery to a subject, i.e., physiologically compatible. Accordingly, pharmaceutical compositions of the processed cells may comprise one or more pharmaceuticall acceptable diluents, excipients or carriers, such as buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. In certain embodiments, the pharmaceutical composition is sterile.

The methods and compositions of the present invention are particularly well-adapted to being practiced using a kit, since they permit the storage and shipment of processed cells or stem cell derived factors. In certain embodiments, a kit comprises a device suitable for administering the processed cell composition, or stem cell derived growth factors, to a subject and containing an amount of stem cell composition or stem cell derived factors to be administered. In one embodiment, a kit useful in the treatment of a musculoskeletal tissue injury in an animal comprises a syringe containing a composition comprising purified adipose tissue-derived stem cells or stem cell derived factors obtained from the animal in a physiologically compatible solution. It is understood that a kit may include any of the purified stem cell populations, stem cell derived growth factors, and compositions described herein. Accordingly, kits of the invention may be prepared for autologous, bob or xenogeneic administration, and may further comprise additional tissue components (cellular or non-cellular) that are co-purified with the stem cells or added to the composition after purification of the stem cells.

### EXAMPLE 1

### Preparation of Adipose-Tissue Derived Stromal Vascular Fractions

Adipose-tissue derived stromal vascular fraction comprising stem cells was prepared from tissue obtained from a horse according to the following procedure.

1. Adipose tissue was trimmed of any visible muscle tissue, lymph nodes or large clots. If the intact tissue was large, it was cut the tissue into multiple pieces. The remaining adipose (about 15-20 grams) was transferred to a 50 mL conical tube(s).

2. The fat was minced in the tube with sterile scissors by opening and closing the scissors while running the blades through the tissue. Most of the remaining fat particles were small, approaching 2 mm in diameter.

3. Sterile PBS was added to each sample conical tube to bring the total volume to 40-45 mL. The conical tube was lidded and mixed by inversion. Using a serological pipette, as much of the fluid as possible was removed and decated into a waste beaker.

4. Step 3 was repeated by adding and removing PBS until the rinsate removed was substantially free of blood (as evidence by red color).

5. An equal volume of digestion cocktail was added to the conical tube. Digestion cocktail was Collagenase and Hyaluronidase in PBS at 0.5-1.5 mg/mL Hyaluronidase with a standard 750-1500 units per mg solid (defined as one unit based on change in absorbance at 600 nM of USP reference standard hyaluronidase) and 2.0 to 3.0 mg/mL Collagenase with a standard PZ activity of 0.1 to 0.2 units per mg solid (defined as 1 U catalyzes the hydrolysis of 1 μmole 4-phenylazobenzyloxycarbonyl-L-prolyl-Lleucyl-glycyl-L-prolyl-D- arginine per minute at 25° C., pH 7.1). The tube was lidded and mixed by inversion.

6. The tube was placed into an aluminum tube holder, which was then placed into a thermal-rocker and incubated for 47-53 minutes with rocking.

7. The volume of the digest was brought to 45-50 mL with PB, and the sample tube was centrifuged (refrigerated to 2-8° C.) at 1,500-1,700 rpm (500-700 g) for 15 minutes.

8. After centrifugation, the supernatant and lipid layer were removed into a sterile container.

9. Approximately 5-10 mL of sterile PBS was added to the tube containing the intact cell pellet, and the bottom of the tube was tapped to resuspend the cell pellet.

10. The resuspended cell pellet was transferred from the sample tube to the top of a cell strainer. The solution was dispensed into the filter to allow the suspension to drain through the filter without overflowing. When the sample was finished straining, the pellet area (5 mL) of the sample tube(s) was rinsed with PBS and the rinsate strained. Thee final volume of the sample was brought to 45-50 mL with PBS. The conical tube was lidded and mixed by inversion.

11. The tube was centrifuged (refrigerated to 2-8° C.) at 1,500-1,700 rpm (500-700 g) for 10 minutes.

12. The supernatant was removed into a sterile container. The bottom of the tube was tapped to resuspend the cell pellet, and the volume was brought to 45-50 mL with sterile PBS. The conical tube was lidded and mixed.

13. The tube(s) was centrifuged (refrigerated to 2-8° C.) at 1,500-1,700 rpm (500-700 g) for 10 minutes.

14. The supernatant was removed into a sterile container. The bottom of the tube was tapped to resuspend the cell pellet, and the volume was brought to 45-50 mL with sterile PBS. The conical tube was lidded and mixed.

15. The tube(s) was centrifuged (refrigerated to 2-8° C.) at 1,500-1,700 rpm (500-700 g) for 10 minutes.

16. The tube was removed from the centrifuge. The supernatant was removed without disturbing the cell pellet.

17. The volume of the cell pellet was brought to a final volume of approximately 2 mL with sterile PBS.

18. Cell count and viability were determined.

### EXAMPLE 2

### Treatment of EIPH Using Adipose-Tissue Derived Stem Cells

A clinical research study was designed as a prospective, un-blinded pilot study to evaluate the effect of intravenous adipose-derived stem cells (ASCs) on EIPH. The EIPH cases included in this study were racing horses or performance horses with tracheobronchoscopy (TBS) and/or bronchoalveolar lavage (BAL) confirmation of blood in the trachea and/or bronchi following strenuous exercise. Adipose tissue samples were collected from each study horse and adipose-derived stromal vascular fraction cells were prepared as described in Example 1 (8 of 12 horses) or stromal vascular fraction cells were cultured using industry standard methods (4 of 12 horses). Adipose-derived stromal vascular fraction cells (containing the ASCs) or cultured ASCs were administered to the horses intravenously in a 10 ml volume (using a Hemonate IV filter to filter out any aggregated cells) two days after the adipose tissue harvest. The amount of adipose-derived stromal vascular fraction cells administered was 2.8-16.5 million nucleated cells, and the amount of cultured ASCs administered were in the range of 15.4-30.8 million stem cells. A second IV treatment with cryo-banked cells was administered 10-14 days after the first treatment at similar dosages. Once the horses were enrolled in the study, no further prophylactic medications for EIPH or inflammatory airway disease (i.e., furosemide, clenbuterol, aminocaproic acid, or corticosteroids) were allowed for the duration of the study.

In order to confirm the diagnosis of EIPH pre-treatment, and to determine the effectiveness of the stem cell treatment post-treatment, a post-race TBS+/−BAL, was performed (1) after a recent race, (2) prior to entering the study, and (3) after the first race post-treatment. The pre- and post-treatment EIPH findings were graded per the EIPH grading scale reported by Hinchcliff et al[12]. Horses remained in light work for 3-5 weeks after the first treatment with stem cells, before they were allowed to return to regular work and racing. Veterinarians and owners/trainers were required to complete pre- and post-treatment data sheets, and submit them for data collection purposes.

Twelve horses have completed the study to date, and all 12 have raced again without any prophylactic medications. A summary of the pre- and post-treatment outcomes of the horses is provided in Table 1. Prior to treatment with ASCs, all 12 of the horses had obvious epistaxis after racing and veterinary confirmation of lung bleeding by TBS (in 8 of 12, others were not examined by endoscope), despite having received pre-race prophylactic medications. All 12 horses were barrel racing quarter horses. Following treatment with ASCs, only 1 of the 12 horses had epistaxis post-race. 5 of the 8 horses that had TBS examinations were confirmed Grade 0 EIPH (no bleeding) by post-race TBS examination. Two horses were confirmed Grade 1 EIPH, and one horse was confirmed Grade 2 EIPH on post-race TBS examinations.

Of the 6 horses with owner or trainer supplied performance evaluations following treatment with ASCs, three of the six horses (50%) performed “significantly better” than before treatment with ASCs, two horses performed “better”, and one horse performed the same as before treatment. All horses raced without prophylactic medications after treatment with ASCs. Owner/trainer reported performance outcomes could have been confounded by other issues such as orthopedic conditions.

There was no difference in clinical outcomes between the 8 horses treated with SVF and the 4 horses treated with the cultured ASCs.

These outcomes, in light of the listed literature, are completely unexpected and dramatically positive. Considering the cellular dose, the size of the horse and the lungs, and the chronicity of this disease, one would not have expected such a large clinical improvement in these horses. Also to consider, is all horses were bleeding pre-treatment in spite of medications, and after treatment were not allowed to have any concurrent medications.

In the recently published 2015 ACVIM Consensus statement on EIPH in horses, EIPH was defined as “the presence of blood detected on tracheobronchoscopic examination after exercise, presence of red blood cells in bronchoalveolar lavage fluid, or both.” The Consensus further states “thus far, interventions to prevent or decrease the severity of EIPH are referred to as prophylaxis and not as treatment”[3]. During the past 4 decades, furosemide has been used prophylactically before racing, to try to prevent EIPH. Studies have shown that furosemide temporarily decreases pulmonary vascular pressures during strenuous exercise, which may decrease the likelihood of capillary stress failure, and therefore reduce the likelihood of hemorrhage in the lungs[1-3, 6]. However, the reduction of bleeding does not reverse the progression of lung tissue damage. With the newer drug rules from multiple jurisdictions coming into play for competition horses, and controversy over the use of furosemide in these horses, furosemide is gradually being phased out as an allowable pre-competition prophylaxis for EIPH[1, 3, 6]. This Example demonstrates that adipose-derived stem cell medical therapy is another option that could provide true repair and regeneration of the damaged tissues rather than just temporary prophylaxis of the problem.

TABLE 1
Summary of pre and post treatment
status of horses treated for EIPH

| Pre Treatment | | Post Treatment | |
|---|---|---|---|
| Prophylactic Drugs | Epitaxis | Prophylactic Drugs | Epistaxis |
| 12/12 | 12/12 | 0/12 | 1/12 |

### EXAMPLE 3

### Treatment of Smoke Inhalation Injury Using Adipose-Tissue Derived Stem Cells

Five horses that suffered severe smoke inhalation due to a large wildfire were treated with stem cells. During evacuation from the fire, two of the horses were abandoned in a trailer and later set free. The other three horses were left in a burning pasture from which they escaped. A complete physical examination including lung auscultation was performed. Lungs sounds on all horses showed wheezes and crackles similar to allergic or pneumonic lungs.

Adipose-derived stem cells in the form of SVF were available for all five horses from prior collections, and all five horses were treated with these cells intravenously. Each horse was treated with its own cells. Table 2 shows the doses administered:

TABLE 2
Doses of SVF Cells Administered

| Horse | Type of Cells | Cell Dose | |
|---|---|---|---|
| 1 | SVF - Frozen dose | 13.78 | million |
| 2 | SVF - Frozen dose | 12.72 | million |
| 3 | Cultured ASC | 4.32 | million |
| 4 | SVF - Frozen dose | 11.56 | million |
| 5 | SVF - Frozen dose | 9.6 | million |

A physical examination was conducted one week and two weeks later on each horse by the attending veterinarian. No further lung abnormalities were noted. The horses resumed normal activity at two weeks post-exposure with no observable impact on health or exercise ability. The horses showed no further health issues over the following year of observation.

The results of this study were surprising, since with this level of inhalation damage, a veterinarian would have expected more long-term effects. Instead, the horses treated with stem cells showed more rapid recovery that horses previously treated by traditional methods. For instance, as described in Kemper et al., J. Am Vet Med Assoc. 1993 Jan. 1; 202 (1):91-4, an earlier study of more traditional treatment methods, five horses were admitted for treatment of smoke-inhalation injuries sustained in a barn fire. Three horses that were mildly affected, with high respiratory rates (24 to 36 breaths/min) and normal to low arterial oxygen tensions (77.0 to 94.1 mm of Hg), responded well to administration of diuretics, bronchodilators, corticosteroids, and antibiotics. However, two horses more severely affected were both were in respiratory distress, with markedly low arterial oxygen tensions (50.4 and 57.1 mm of Hg) and cyanosis. These two horses were treated with fluid resuscitation in addition to the treatments given to the less severely affected horses. Tracheostomy was performed to facilitate removal of large, obstructive, pseudomembranous tracheobronchial casts. Oxygen was administered by nasal or tracheal insufflation or by use of a high-frequency jet ventilator. The most severely affected horse developed hemorrhagic colitis and was euthanatized. The four surviving horses recovered in 2 to 5 months and resumed working without reduction in performance capability.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

All publications and patent applications described herein are hereby incorporated by reference in their entireties.

### REFERENCES

1. Birks E K, Shuler K M, Soma L R, Martin B B, Marconato L, Del Piero F, et al. EIPH: postrace endoscopic evaluation of Standardbreds and Thoroughbreds. Equine Vet J Suppl. 2002:375-8.
2. Derksen F, Williams K and Stack A. Exercise-induced pulmonary hemorrhage in horses: the role of pulmonary veins. Compend Contin Educ Vet. 2011; 33:E6.
3. Hinchcliff K W, Couetil L L, Knight P K, Morley P S, Robinson N E, Sweeney C R, et al. Exercise induced pulmonary hemorrhage in horses: American College of Veterinary Internal Medicine consensus statement. J Vet Intern Med. 2015; 29:743-58.
4. Kindig C A, McDonough P, Fenton G, Poole D C and Erickson H H. Efficacy of nasal strip and furosemide in mitigating EIPH in Thoroughbred horses. J Appl Physiol (1985). 2001; 91:1396-400.
5. Manohar M and Goetz T E. Pulmonary vascular pressures of exercising thoroughbred horses with and without endoscopic evidence of EIPH. J Appl Physiol (1985). 1996; 81:1589-93.
6. Sullivan S and Hinchcliff K. Update on exercise-induced pulmonary hemorrhage. Vet Clin North Am Equine Pract. 2015; 31:187-98.
7. Sullivan S L, Anderson G A, Morley P S and Hinchcliff K W. Prospective study of the association between exercise-induced pulmonary haemorrhage and long-term performance in Thoroughbred racehorses. Equine Vet J. 2015; 47:350-7.
8. Michelotto P V, Jr., Muehlmann L A, Zanatta A L, Bieberbach E W, Kryczyk M, Fernandes L C, et al. Pulmonary inflammation due to exercise-induced pulmonary haemorrhage in Thoroughbred colts during race training. Vet J. 2011; 190:e3-6.
9. Morris M E, Beare J E, Reed R M, Dale J R, LeBlanc A J, Kaufman C L, et al. Systemically delivered adipose stromal vascular fraction cells disseminate to peripheral artery walls and reduce vasomotor tone through a CD11b+ cell-dependent mechanism. Stem Cells Transl Med. 2015; 4:369-80.
10. Rigotti G, Marchi A, Galie M, Baroni G, Benati D, Krampera M, et al. Clinical treatment of radiotherapy tissue damage by lipoaspirate transplant: a healing process mediated by adipose-derived adult stem cells. Plast Reconstr Surg. 2007; 119:1409-22; discussion 23-4.
11. Traktuev D O, Merfeld-Clauss S, Li J, Kolonin M, Arap W, Pasqualini R, et al. A population of multipotent CD34-positive adipose stromal cells share pericyte and mesenchymal surface markers, reside in a periendothelial location, and stabilize endothelial networks. Circ Res. 2008; 102:77-85.
12. Hinchcliff K W, Jackson M A, Brown J A, Dredge A F, O'Callaghan P A, McCaffrey J P, et al. Tracheobronchoscopic assessment of exercise-induced pulmonary hemorrhage in horses. Am J Vet Res. 2005; 66:596-8.

1. A method for treating or preventing an injury or disease associated with damage to blood vessels, inflammation, and/or fibrosis in a mammal in need thereof, comprising providing to the mammal an effective amount of a pharmaceutical composition comprising one or more stem cells, or one or more stem cell derived factors.

2. The method of claim 1, wherein the mammal is a horse, human, camel or dog.

3. The method of claim 1 or claim 2, wherein the stem cells were obtained from the mammal.

4. The method of claim 1 or claim 2, wherein the stem cells were obtained from a donor animal.

5. The method of any of claims 1-4, wherein the pharmaceutical composition comprises a stromal vascular fraction comprising stem cells.

6. The method of any of claims 1-4, wherein the pharmaceutical composition comprises isolated stem cells.

7. The method of any of claims 1-6, wherein the stem cells are derived from adipose tissue.

8. The method of any one of claims 1-7, wherein the pharmaceutical composition is administered intravenously, orally, nasally, or by inhalation.

9. The method of any one of claims 1-8, wherein the pharmaceutical composition further comprises one or more additional active agent for the treatment of the lung injury.

10. The method of claim 9, wherein the one or more additional active agent comprises a non-steroidal anti-inflammatory agent or a steroid.

11. The method of any one of claims 1-10, wherein the injury or disease is an injury or disease of a lung.

12. The method of claim 11, wherein the lung injury is exercise induced pulmonary hemorrhage (EIPH), chronic obstructive pulmonary disorder (COPD), lung fibrosis, smoke inhalation, other toxic inhalation, or pneumonia infection.

13. The method of any one of claims 1-12, wherein the mammal is provided with at least one dose of the pharmaceutical composition, wherein the dose comprises less than 15 million stem cells.

14. The method of claim 13, wherein the dose comprises less than 10 million stem cells.
